(19) United States
(12) Patent Application Publication
(10) Pub. No.: US 2006/0074277 A1
(43) Pub. Date: Apr. 6, 2006

(54) Retractor

(76) Inventor: Kazuki Yoshida, Kanzaki-gun (JP)

Correspondence Address: MORGAN & FINNEGAN, L.L.P., 3 World Financial Center, New York, NY, 10281-2101, US

(21) Appl. No.: 11/244,504

(22) Filed: Oct. 5, 2005

(30) Foreign Application Priority Data
Oct. 6, 2004 (JP) 2004-294026

(51) Int. Cl. A61B 1/32

(52) U.S. Cl. 600/209

(57) ABSTRACT

The object of the present invention is to provide a retractor with a remarkably simple structure which can be adapted to and used for different sites of living tissues. The retractor 10 is provided with a holding part 12 for holding a living tissue, and holding part 12 is comprised of a movable member 16 capable of a forward-and-backward movement protruding from the distal end part of rod-shaped support member 14 and at least one pair of flexible strip-shaped members 18 and 20 whose one end is fixed to movable member 16, wherein strip-shaped members 18 and 20 expand into an extended condition at the advancing-limit position of movable member 16, and pair of strip-shaped member 18 and 20 form an asymmetric curved shape relative to the movable member 16 at a given position of movable member 16 backward from such advancing-limit position.

### TECHNICAL FIELD

The present invention relates to a retractor for holding a part of or the whole organ such as liver, lung, esophagus, stomach, intestines, kidney, uterus, ovary and the like, during, for example, a surgery.

### BACKGROUND ART

Conventionally, as for a retractor for holding a living tissue, the followings have been known:

a retractor comprising a handle; a support shaft extending from the handle; a tube provided on the support shaft such that it is longitudinally slidable against the support shaft; and an expandable frame provided at the end of the support shaft, connected to the tube, providing a platform to manipulate a tissue by expanding according to the movement of the tube against the support shaft; wherein the frame comprises a pair of expandable fingers; each of the fingers comprises a first and a second outer flexible strips fixed to the end of the tube, and a first and a second inner flexible strips fixed to the end of the support shaft; the first outer flexible strip and the first inner flexible strip are joined at the first joining part at the distal end of the finger; the second outer flexible strip and the second inner flexible strip are joined at the second joining part at the distal end of the finger; the first joining part and the second joining part are not joined; and the fingers bend outwardly in the opposite lateral direction according to the movement in the shaft direction of the tube against the shaft, to form a platform to hang a tissue (see Japanese Patent Publication No. 3391824), and

a surgical retractor comprising a handle means including an operative structure; and a housing means set on the operative structure of the handle means, wherein the housing means comprises at least one central element which is provided adjacent to a guiding element capable of moving forward and backward relatively with the central element, and the central element and the guiding element include a proximal end and a distal end. The surgical retractor further comprises a retractor assembly including a reciprocal yoke assembly having a slide yoke means and a pivot yoke means, and a plurality of collapsible retractor blades which are mutually connected by pivot pins. The slide yoke means and the pivot yoke means cooperate with the housing means so that the relative-mutual movement in the shaft direction of the central element and the guiding element is useful for moving the collapsible retractor blades between their closed position and their spread position (see Japanese Patent Publication No. 3369219). In addition to those mentioned above, a various retractors have been proposed (see US Patent Publication Nos. U.S. Pat. Nos. 5,467,763, 5,803,902, 4,909,789, 5,235,966, 5,325,848, 349,339, 5,318,586, 5,439,476, 699,358, 5,339,803, 5,405,360, 5,441,044, 5,865,802 and 5,860,987).

The retractor of Japanese Patent Publication No.3391824, however, is operated under a multiple-leaf-spring system, and when pressing a tissue, the metal blades get thinner and exhibit an unstable shape due to inadequate rigidity. Particularly, as the metal turn-up portions have discontinuous surfaces and are edged, it happens to be dangerous by damaging the tissues, and therefore it is out of market currently. The retractor of Japanese Patent Publication No. 3369219, when there is a gap between the metal parts on the distal end and it moves forward in the shaft direction, may involve a risk of injuring a tissue by the distal end of one of the metal parts. Further, the blades, when closing, may also trap a tissue between their gaps as if to cut the tissue with scissors as the apparatus further closes. In addition, the retractor of US Patent Publication No. U.S. Pat. No. 5,467,763 converts into a given form by means of a large number of metal flexions provided, while a careful handling is required because when rotating the apparatus, the metal part on the distal end may involve a risk of penetrating a tissue.

Further, the retractor of US Patent Publication No. U.S. Pat. No. 5,803,902, for a safer and more effective performance, is wrapped in a sheath, thus creating a disadvantage that its diameter becomes large when folding the sheath. It, therefore, cannot be expected to achieve a finer diameter to serve as an even less-invasive apparatus. Additionally, since the shape of the sheath in the most expanded state is fixed from the beginning, the retractor will remain in a limited shape. Therefore, its uses are limited. The retractor of the US Patent Publication No. U.S. Pat. No. 4,909,789 is constituted of three, four, and six tubular rods respectively for its distal end shape, and of a fine bulb at a farthest end part. Since the plurality of tubular round rods are independent of one another, when pressing a tissue, the distal end of the plurality of rods fail to create a flat surface thus it cannot be expected to have a tissue-holding effect. Further, the distal end part of the apparatus involves a risk of penetrating a tissue when moved forward in the shaft direction. The retractor of US Patent Publication No. U.S. Pat. No. 5,235,966 is shaped into a bifurcated form to the left and right on the distal end. Such distal end is comprised of three parts, wherein two pairs of hinges are connected at the central part to the left and right semi-tubular rods. Although the flat surface presses a tissue effectively, the retractor involves a risk of injuring a tissue when moving forward on the shaft and, of cutting the tissue that has been trapped between the three parts and two hinges when closing the apparatus.

The retractor of US Patent Publication No. U.S. Pat. No. 5,325,848 is connected with a semi-circular tubular rod at two places by pins on the right and left side of the central shaft. Plural semi-circular tubular rods are present in the backward direction so as to create the least possible gaps in-between in order to press a tissue effectively by a flat surface. The retractor, however, is comprised of a large number of constituent parts, thus creating a large diameter, and therefore it cannot be expected to achieve a finer diameter to serve as an even less-invasive apparatus. The retractor of US Patent Publication No. U.S. Pat. No. 349,339 simply presses a tissue perpendicularly with two pairs of thin bands provided on the shaft of the apparatus, thus failing to create a pressing effect of a flat surface. Therefore, its uses are limited. The retractors of US Patent Publication No. U.S. Pat. No. 5,318,586 and US Patent Publication No. U.S. Pat. No. 5,439,476 are capable of pressing a tissue with a flat surface without any gaps by expanding a balloon from the center of the apparatus, while it is disadvantageous in that it becomes slippery as no friction is generated when the direction is deviated.

The retractor of US Patent Publication No. U.S. Pat. No. 699,358, similar to the retractor of US Patent Publication No. U.S. Pat. No. 349,339, simply presses a tissue perpendicularly with two pairs of thin bands provided on the shaft of the apparatus, thus failing to create a pressing effect of a flat surface. Therefore, its uses are limited. Further, there is a flexion point present in the middle point of the blade. The retractor of US Patent Publication No. U.S. Pat. No. 5,339,803 comprises a pair of blades extending from backward of the shaft of the apparatus, to the left and then through the center of front distal end and bifurcating to the right and then back to the original position. A V-shaped support is present for enhancing the rigidity of the blade, extending from the farthest end of the apparatus to the middle points of the pair of blades. The connection points of the V-shaped support have a disadvantageous structure in that it is difficult to fold the blades and that they can be damaged by the outer casing particularly at the time of storing, and thus it could drop the broken pieces in a human body. The retractor of US Patent Publication No. U.S. Pat. No. 5,405,360 only has a function of pressing a tissue in the shaft direction by five points with a piece of meshed cloth thereon. For this reason, it is unrealistic to alter the apparatus for a finer diameter. The retractor of US Patent Publication No. U.S. Pat. No. 5,441,044 has a structure of a semi-circle, leaf-spring ring with a membrane filling the gap, and is a simple apparatus with a limited use. It is, therefore, incapable of pressing a small tissue. The retractor of US Patent Publication No. U.S. Pat. No. 5,865,802 operates by an expandable balloon system including a built-in metal framework, which is highly effective for holding a tissue. Meanwhile, its disadvantage is that it is slippery when the direction is deviated. Also, it is unrealistic to alter the retractor for a finer diameter as it has a complicated structure. The retractor of US Patent Publication No. U.S. Pat. No. 5,860,987 is a simple apparatus with a platy ring whose use is limited to holding of a tissue. It is incapable of pressing a small tissue.

The object of the present invention is to provide a retractor with a remarkably simple structure which can be adapted to and used for different sites of living tissues, and also to provide a retractor with conventional retractor functions, which is practical for other uses at the same time.

As a result of a keen study to solve the above-mentioned problems, the present inventors have found that by using extendable strip-shaped members in a holding part and thus rendering the structure of the apparatus remarkably simple, the diameter of the retractor itself at the time of insertion through a port can be reduced, and further, that by making the holding part form an asymmetric shape and the movable member move in a full stroke in order to change the shape of the holding part, the retractor can be adapted to and used for different sites of living tissues. The present inventors have also found out that the retractors can have not only conventional-retractor functions but also other functions, and thus the present invention has been completed.

### DISCLOSURE OF THE INVENTION

The present invention relates to a retractor provided with a holding part for holding a living tissue, wherein the holding part comprises a movable member capable of a forward-and-backward movement protruding from the end part of a rod-shaped support member, and at least a pair of flexible strip-shaped members (band-like members) whose one end is fixed to the movable member; and wherein the strip-shaped members expand into an extended condition at the advancing-limit position of the movable member, and the pair of strip-shaped members form an asymmetric shape relative to the movable member at a given position of the movable member backward from the advancing-limit position (“1”), the retractor according to “1” wherein the movable member is capable of moving in a full stroke (“2”), a retractor provided with a holding part for holding a living tissue, wherein the holding part comprises a movable member capable of moving forward and backward in a full stroke, protruding from the end part of the rod-shaped support member, and a flexible strip-shaped member whose one end is fixed to the movable member, wherein the strip-shaped members expand into an extended condition at the advancing-limit position of the movable member, and the pair of strip-shaped members form an asymmetric shape relative to the movable member at a given position of the movable member backward from the advancing-limit position (“3”), and the retractor according to “3”, wherein the strip-shaped members are at least one pair of strip-shaped members, and the pair of strip-shaped members form an asymmetric curved shape relative to the movable member at a given position of the movable member from the advancing-limit position (“4”).

The present invention also relates to the retractor according to any one of “1” to “4”, wherein the rod-shaped support member and/or the movable member is rotatable (“5”), the retractor according to any one of “1” to “5”, comprising a locking means capable of locking the movable member at a given position (“6”), the retractor according to any one of “1” to “6”, wherein a bulb-shaped member is provided at the distal end of the holding part (“7”), the retractor according to any one of “1” to “6”, wherein an electrode is provided at the distal end of the holding part (“8”), and the retractor according to any one of “1” to “6”, provided with a suction opening capable of suctioning body fluid (“9”).

The present invention further relates to a retractor provided with a holding part for holding a living tissue, wherein an electrode is provided at the distal end of the holding part (“10”), and a retractor provided with a holding part for holding a living tissue, comprising a suction opening capable of suctioning body fluid (“11”).

The retractor of the present invention is of a remarkably simple structure and can be adapted to and used for different sites of living tissues. In addition, the retractor of the present invention not only has the conventional retractor functions but also is practical for other uses.

### BRIEF DESCRIPTION OF DRAWINGS

FIG. 1 is a front view of the retractor of the present invention.

FIG. 2 is a set of graphics illustrating a modified embodiment of the retractor shown in FIG. 1.

FIG. 3 is a set of graphics illustrating how the retractor shown in FIG. 1 is used.

FIG. 4 is a set of graphics illustrating how the retractor shown in FIG. 1 is used.

FIG. 5 is a set of graphics illustrating how the retractor shown in FIG. 1 is used.

FIG. 6 is a set of graphics showing another example of the retractor of the present invention.

FIG. 7 is a set of graphics showing another example of the retractor of the present invention.

FIG. 8 is a set of graphics showing another example of the retractor of the present invention.

FIG. 9 shows another example of the retractor of the present invention.

FIG. 10 shows another example of the retractor of the present invention.

### BEST MODE OF CARRYING OUT THE INVENTION

A retractor of the first invention includes any retractor, as long as it is a retractor provided with a holding part for holding a living tissue, wherein the holding part comprises movable member capable of a forward-and-backward movement protruding from the end part of a rod-shaped support member, and at least a pair of flexible strip-shaped members whose one end is fixed to the movable member; and wherein the strip-shaped members expand into an extended condition at the advancing-limit position of the movable member, and the pair of strip-shaped members form an asymmetric shape relative to the movable member at a given position of the movable member backward from such advancing-limit position. According to the retractor of the present invention, it is possible to hold (press) a remarkably extensive area of tissue with the whole holding part, and also different areas of tissue with one side of the holding part, by rotating the shaft by 180°, since the holding part is an asymmetric type. In other words, the retractor of the present invention can be used as different sizes of retractors, without changing apparatuses. Moreover, as the holding part comprises a movable member capable of a forward-and-backward movement protruding from the end part of the rod-shaped support member, and as the pair of strip-shaped members are of a construction to form an asymmetric, curved shape relative to the movable member at a given position of the movable member backward from such advancing-limit position, the forward-and backward movement of the movable member enables the strip-shaped members to form various curved shapes and thus to convert into optimum shapes conforming to the shape of the subject tissue and the use, to adapt to various sites of living tissues. Meanwhile, it is preferable that the movable member is capable of moving in a full stroke over the end of the rod-shaped support member, allowing the formation of an even wider range of curved shapes.

Further, in the retractor of the present invention, a pair of strip-shaped members form a (asymmetric) curved shape relative to the movable member, creating gaps (space) between the strip-shaped members and the movable member and allowing direct observation of a tissue through the gaps, and incision or coagulation of the tissue with other apparatuses through the gaps. The gaps also enable the holding part to hold a living tissue more stably by trapping the tissue and thus prevent sliding. In addition, because of their flexibility (bendability), the strip-shaped members can fit to the tissue even if they are inserted into the desired position in a somewhat deviated angle. As a result, there is almost no limitation as to the angles from a port, whereby approach is allowed and facilitated from any ports.

The rod-shaped support member is a rod-shaped member made of stainless steel and the like, which is, for example, circular or polygonal shape in cross section, the diameter being preferably about 3-15 mm, and more preferably about 3-7 mm. The retractor of the present invention not only causes the strip-shaped members to expand into an extended condition at the advancing-limit position of the movable member, but also has a remarkably simple structure. Consequently, by reducing the diameter of the rod-shaped support member as mentioned above, the diameter of the retractor itself can also be reduced. Thus, the retractor can be inserted through a port of a fine diameter, permitting an even less-invasive surgery.

Further, the retractor of the present invention is provided with a movable member capable of a forward-and-backward movement protruding from the end part of the rod-shaped support member. Such movable member includes, for example, a rod-shaped member which is inserted through the interior of the rod-shaped support member and, more specifically, for example, a rod-shaped member made of stainless steel and the like, with a certain rigidity, wherein the diameter of the portion forming the holding part protruding from the rod-shaped support member is about 0.5-3 mm, and preferably about 0.5-1.5 mm. As a result, the movable member can cooperate with the strip-shaped member for a stable holding of a living tissue. Meanwhile, it is preferable to give a large diameter to the portion other than the one forming the holding part, in order to enhance stability of the movable member.

Furthermore, at least one pair of strip-shaped members are provided on both sides of the movable member. The strip-shaped members are, for example, about 5-15 mm and preferably about 3-7 mm in width, about 0.5-3 mm and preferably about 0.5-1.5 mm in thickness, and about 3-15 cm and preferably about 5-10 cm in length. The materials are not limited as long as they are flexible, including, for example, metal materials and synthetic resins such as polyethylene, polypropylene, and synthetic rubber, while high-density polyethylene is particularly preferable. Complex materials such as a combination of a metal material and a synthetic resin material, and of a carbon fiber and a synthetic resin material may also be used. An asymmetric curved shape can be formed by using strip-shaped members of different length. The retractor essentially has to be provided with at least one pair of, i.e. two strip-shaped members, while it may be provided with three strips or four strips (two pairs) or more. Also, the strip-shaped members essentially have to be fixed to the movable member at one end, while the other end of the strip-shaped member may be fixed to the rod-shaped support member or to a movable member different from the one which the one end is fixed to. Moreover, the strip-shaped members forming the holding part may be constituted such that their length can be altered independently or together, which can be exemplified by a constitution wherein the other end side of the strip-shaped members is capable of moving into or out of the interior of the rod-shaped support member. Consequently, the holding part can be converted into more optimum shapes conforming to the shape of the subject tissue and the use, to adapt to various sites of a living body.

The retractor of the second invention includes any retractor as long as it is a retractor provided with a holding part for holding a living tissue, wherein the holding part comprises a movable member capable of moving forward and backward in a full stroke, protruding from the end part of a rod-shaped support member, and a flexible strip-shaped member whose one end is fixed to the movable member; and wherein the strip-shaped member expands into an extended condition at the advancing-limit position of the movable member, and the pair of strip-shaped member forms a curved shape relative to the movable member at a given position of the movable member backward from such advancing-limit position. According to the retractor of the present invention, the movable member is capable of moving in a full stroke and changing diversely the curved shape formed by the strip-shaped member. Therefore, the holding part can be converted into optimum shapes conforming to the shape of the subject tissue and the use, to adapt to various sites of a living body.

Further, in the retractor of the present invention, the strip-shaped members form a curved shape, creating a gap (space) between the strip-shaped member and the movable member, allowing direct observation of a tissue through the gap, and incision or coagulation of the tissue with other apparatuses through the gap. The gap also enables the holding part to hold a living tissue more stably by trapping tissues and thus prevent sliding. In addition, because of its flexibility (bendability), the strip-shaped member can fit to the tissue even if it is inserted into the desired position in a somewhat deviated angle. As a result, there is almost no limitation as to the angles from a port, whereby approach is allowed and facilitated from any ports.

The rod-shaped support member is a rod-shaped member made of stainless steel and the like, which is, for example, circular or polygonal shape in cross section, the diameter being preferably about 3-15 mm, and more preferably about 3-7 mm. The retractor of the present invention not only causes the strip-shaped member to expand into an extended condition at the advancing-limit position of the movable member, but also has a remarkably simple structure. Consequently, by reducing the diameter of the rod-shaped support member as mentioned above, the diameter of the retractor itself can also be reduced. Thus the retractor can be inserted through a port with a fine diameter.

The retractor of the present invention is provided with a movable member capable of moving forward and backward in a full stroke over the end of the rod-shaped support member. Such movable member includes, for example, a rod-shaped member which is inserted through the interior of the rod-shaped support member and, more specifically, for example, a rod-shaped member made of stainless steel and the like, with a certain rigidity, wherein the diameter of the portion forming the holding part protruding from the rod-shaped support member is about 0.5-3 mm, and preferably about 0.5-1.5 mm. As a result, the movable member can cooperate with the strip-shaped member for a stable holing of a living tissue. Meanwhile, it is preferable to give a large diameter to the portion other than the one forming the holding part, in order to enhance stability of the movable member.

Furthermore, the movable member is provided with a strip-shaped member on its one side or both sides. If the strip-shaped members are provided on both sides of the movable member, such one pair of strip-shaped members may form a symmetric curved shape relative to the movable member at a given position of the movable member backward from the advance-limit position, while it is preferable that they form an asymmetric curved shape. Thus, it is possible to hold (press) a remarkably extensive area of tissue with the whole holding part, and also to hold different areas of tissue with one side of the holding part, by rotating the shaft by 180°, since the holding part is asymmetric type. In other words, the retractor of the present invention can be used as three different sizes of retractors without changing apparatuses. Meanwhile, an asymmetric curved shape can be formed, for example, by using strip-shaped members of different length.

Furthermore, the strip-shaped member include, for example, a strip-shaped member of about 5-15 mm and preferably 3-7 mm in width, about 0.5-3 mm and preferably about 0.5-1.5 mm in thickness, and about 3-15 cm and preferably about 5-10 cm in length. The materials are not limited as long as they are flexible, including, for example, resins such as polyethylene, polypropylene, and synthetic rubber, while high-density polyethylene is particularly preferable. The retractor may be provided with one strip-shaped member while it may be provided with two (a pair) or four strips (two pairs) or more. Also, the strip-shaped member essentially has to be fixed to the movable member at one end, while the other end of the strip-shaped member may be fixed to the rod-shaped support member or to a movable member different from the one which the one end is fixed to. Moreover, the strip-shaped members forming the holding part may be constituted such that their lengths can be altered independently or together, which can be exemplified by a constitution wherein the other end side of the strip-shaped members is capable of moving into or out of the interior of the rod-shaped support member. Consequently, the holding part can be converted into more optimum shapes conforming to the shape of the subject tissue and the use, to adapt to different sites of a living body.

The preferred rod-shaped support member and/or movable member of the retractors of the first and the second invention is rotatable, whereby the strip-shaped members can be converted into an even wider range of shapes, leading to a more extensive range of use. Also, it is preferable that the retractors include a locking means capable of locking the movable member at a given position, whereby the movable member can be locked at any desired advanced or withdrawn position, and also capable of fixing rotation position when the movable members are rotatable.

Further, the preferred holding part of the retractor of the first and the second invention is, at its distal end (distal end of the movable member), provided with a bulb-shaped member and electrodes. The bulb-shaped member is, for example, about 2-10 mm, and preferably about 3-7 mm in diameter, the material including metal such as stainless-steel and synthetic resin, while the preferred material is the same resin used for the strip-shaped member since it facilitates the production. By providing the bulb-shaped member, the retractor is prevented from injuring a living tissue and also from sliding, thus allowing a stable holding of a living tissue. Furthermore, the electrode includes spicular, rod-shaped, or bulb-shaped electrodes and the like and, more specifically, a female electrode (monopolar electrode) in a monopolar electrode form wherein a counter-electrode plate is combined with a female electrode, and an electrode in multipolar form wherein the tips of forceps constitute a pair of electrodes. By providing an electrode, a tissue can be coagulated and incised while being held (pressed). Therefore, the simultaneous procedure is possible without the need for placing apparatuses such as an electric cautery and the like into and out of a body. As a result, a less-invasive surgery with one fewer cuts on the body is can be performed.

Moreover, it is preferable that the retractors of the first and the second invention are provided with a suction opening capable of suctioning body fluid. The suction opening capable of suctioning body fluid can be provided at the distal end part of the rod-shaped support member by, for example, inserting the suction tube through the interior of the rod-shaped support member, whereby body fluid can be suctioned and washed away while holding the tissue. This contributes to eliminate one port, permitting to perform a less-invasive surgery with one fewer cuts on the body. In particular, in a constitution wherein the movable member is capable of moving forward and backward in a full stroke, the distal end part of the holding part (distal end part of the movable member) and the suction opening provided at the distal end part of the rod-shaped support member become remarkably close to each other, permitting the effective suctioning of body fluid.

Furthermore, the retractor of the third invention includes any retractor as long as they are provided with a holding part for holding a living tissue, and an electrode at the distal end of the holding part. It can be exemplified by the retractors of the first and the second invention provided with an electrode at the distal end part of the holding part (distal end part of the movable member). According to the retractor of the present invention, a tissue can be coagulated and incised while being held (pressed). Therefore, the simultaneous procedure is possible without the need for placing apparatuses such as an electric cautery and the like into and out of the body. As a result, a less invasive surgery with one fewer cuts on the body can be performed. The electrode includes spicular, rod-shaped, or bulb-shaped electrodes and the like and, more specifically, a female electrode (monopolar electrode) in a monopolar electrode form wherein a counter-electrode plate is combined with a female electrode, and an electrode in multipolar form wherein the tips of forceps constitute a pair of electrodes.

Still further, the retractor of the forth invention includes any retractor as long as they are retractors provided with a holding part capable of holding a living tissue, wherein a suction opening capable of suctioning body fluid is placed. The suction opening capable of suctioning body fluid can be provided at the distal end part of the rod-shaped member by, for example, inserting a suction tube through the interior of the rod-shaped support member of the retractors of the first and the second invention, whereby body fluid can be suctioned and washed away while holding the tissue. This contributes to eliminate one port, permitting to perform a less-invasive surgery with one fewer cuts on the body. In particular, in a constitution wherein the movable member is capable of moving forward and backward in a full stroke, the distal end part of the holding part (distal end part of the movable member) and the suction opening provided at the distal end part of the rod-shaped support member become remarkably close to each other, permitting the effective suctioning of body fluid.

A more detailed explanation of the present invention is provided in the following description with reference to the following figures, while the technical scope of the present invention is not limited to these examples.

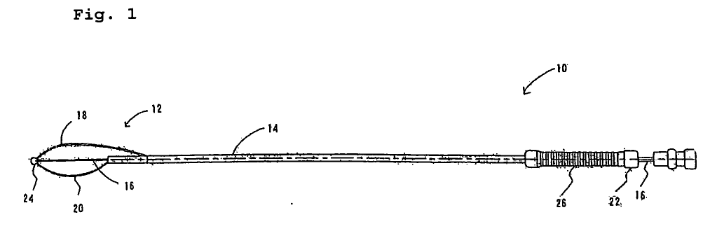

As illustrated in FIG. 1, according to an example of the present invention, retractor 10 is a retractor provided with a holding part 12 for holding a living tissue. Holding part 12 includes: a movable member 16 capable of moving forward and backward in a full stroke, protruding from the end part of rod-shaped support member 14 which is about 5 mm in diameter and about 50 cm in length; and a pair of strip-shaped member 18 and 20 made of high-density polyethylene, whose one end is fixed to the distal end of the movable member 16, and the other end to the rod-shaped support member 14. As for the pair of strip-shaped members 18 and 20, strip-shaped member 18 is longer than strip-shaped member 20, so that they can form an asymmetric shape relative to the rod-shaped support member 14 at a given position of the movable member 16 backward from the advancing-limit position.

Movable member 16 is inserted within rod-shaped support member 14 and connected to locking means 22. This locking means 22 locks movable member 16 at a given position such that the holding part 12 can be kept in a given (asymmetric) shape. Further, the diameter of the retractor is reduced into the possible smallest size when the movable member 14 is at the advancing-limit position, causing strip-shaped members 18 and 20 to expand into an extended condition. Thus, the retractor can be introduced through a fine port, permitting a less-invasive surgery. Also, a bulb-shaped member 24, which is about 5 mm in diameter and of the same material that is used for strip-shaped members 18 and 20, is provided at the distal end of movable member 16. This bulb-shaped part 24 prevents the retractor from injuring a tissue as well as sliding on a tissue. Meanwhile, numeral 26 indicates a gripping member.

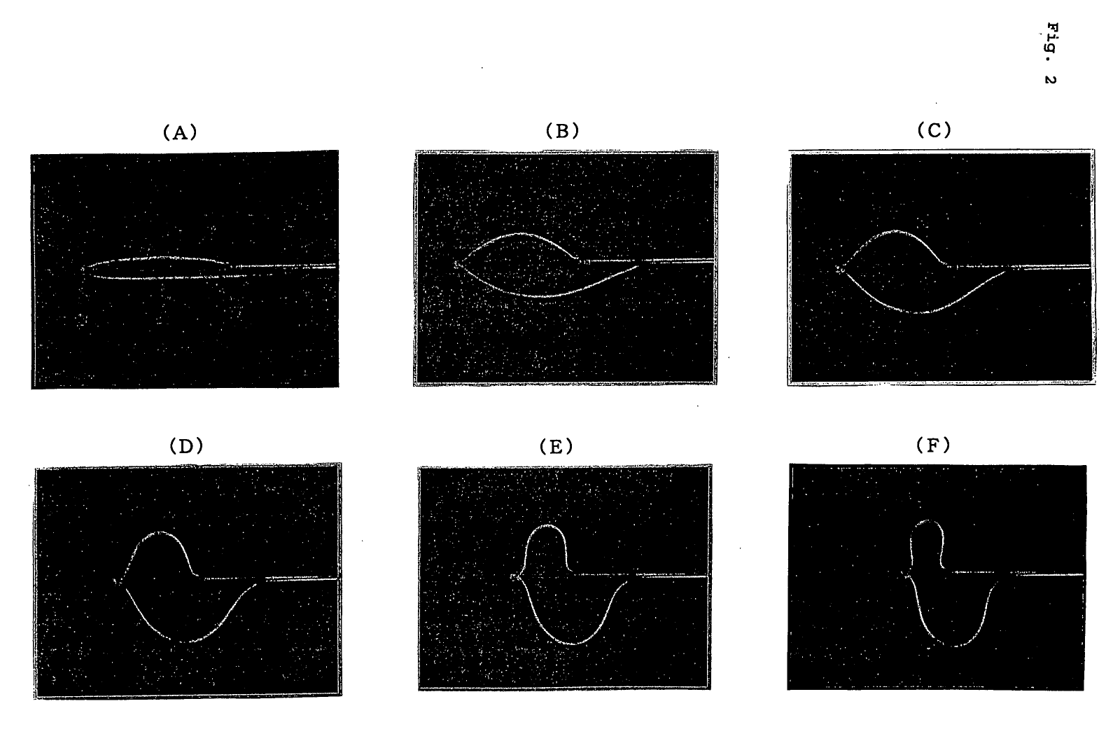

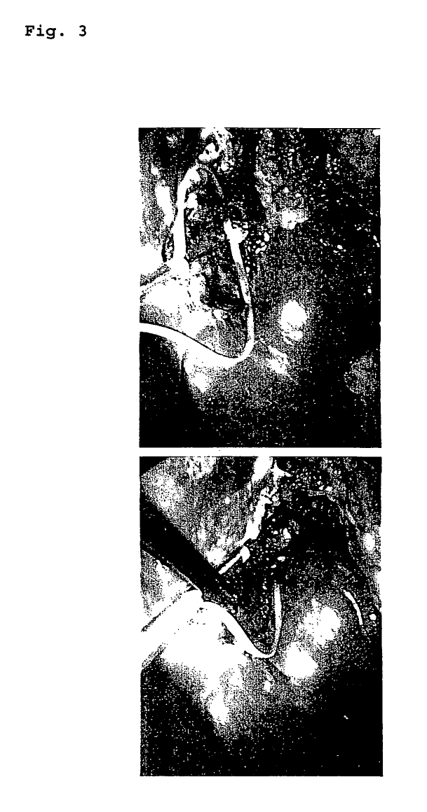

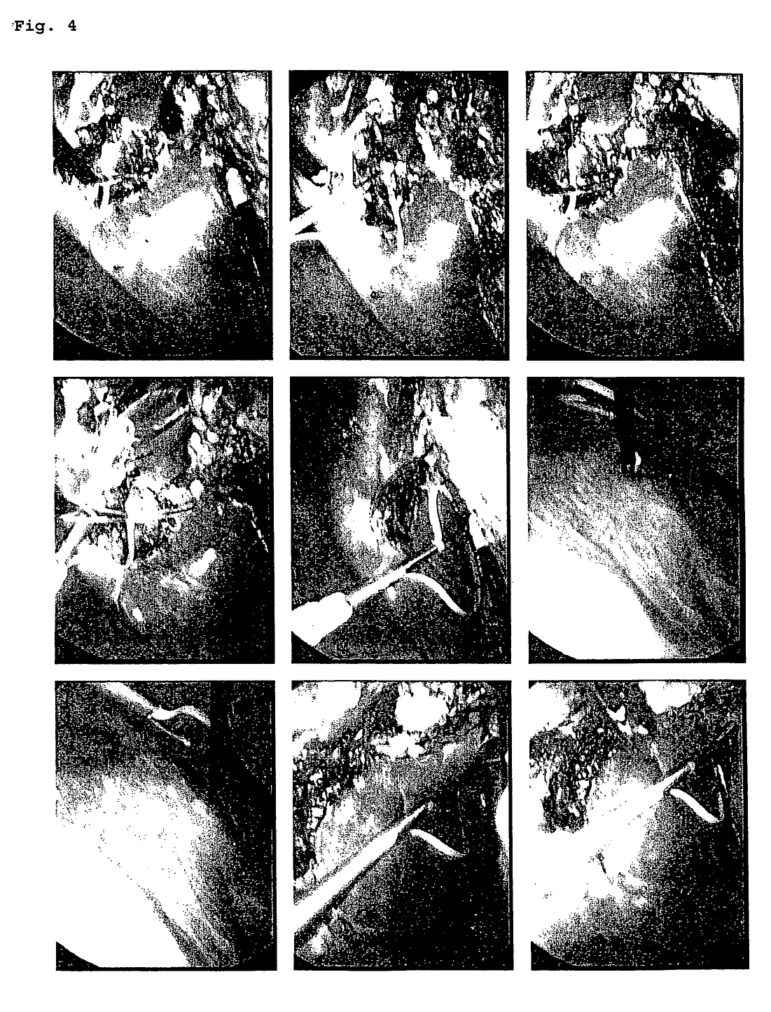

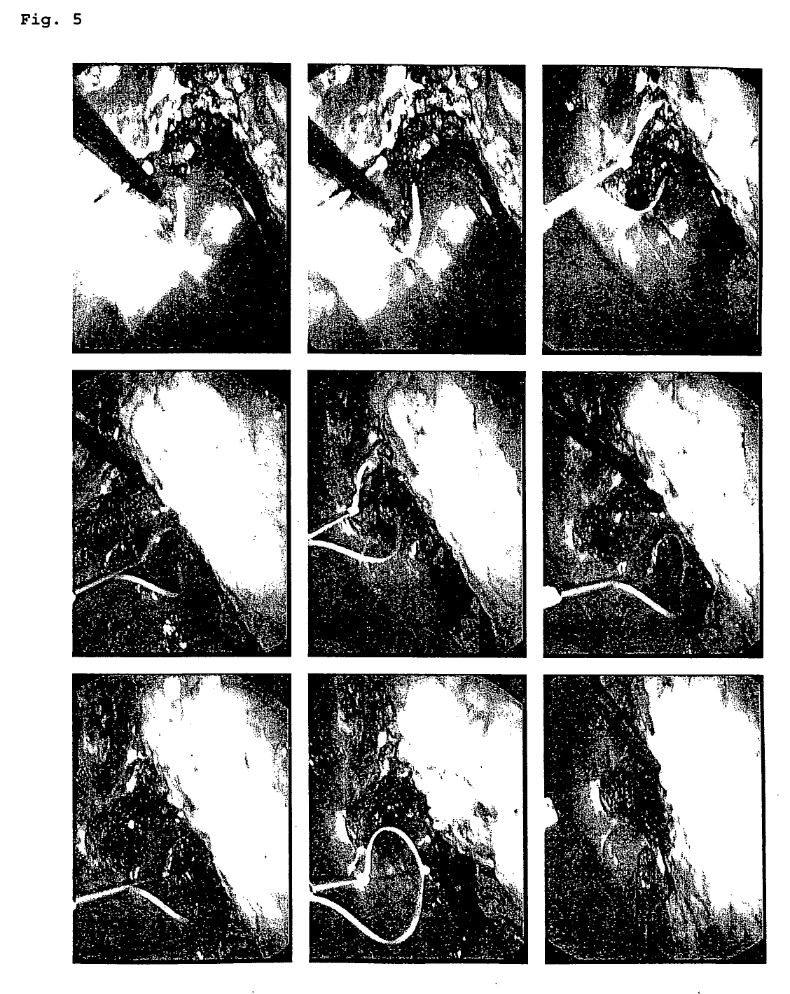

When using the above-mentioned retractor 10, as shown in FIG. 2 (A), it is inserted into a port with the movable member 14 locked at the advancing-limit position and strip-shaped members 18 and 20 in the extended condition. Subsequently, according to the intended use, the movable member 14 is withdrawn, and locked by locking member 22 so that a pair of strip-shaped member 18 and 20 form a given asymmetric shape (see FIG. 2 (B)-(F)), and then the retractor will be used during the surgery for holding a living tissue (see FIGS. 3-5).

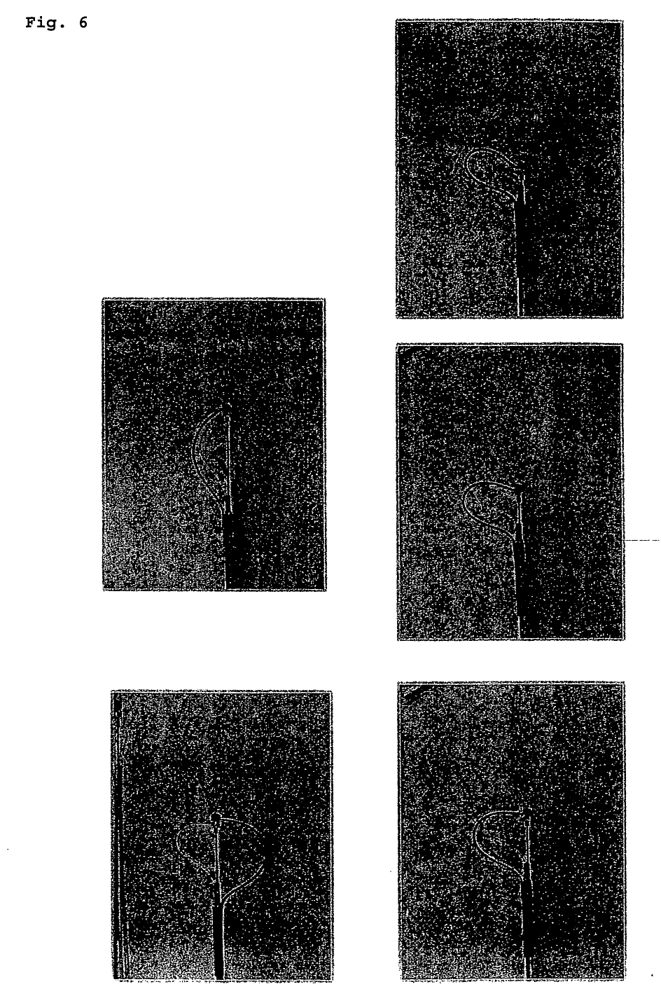

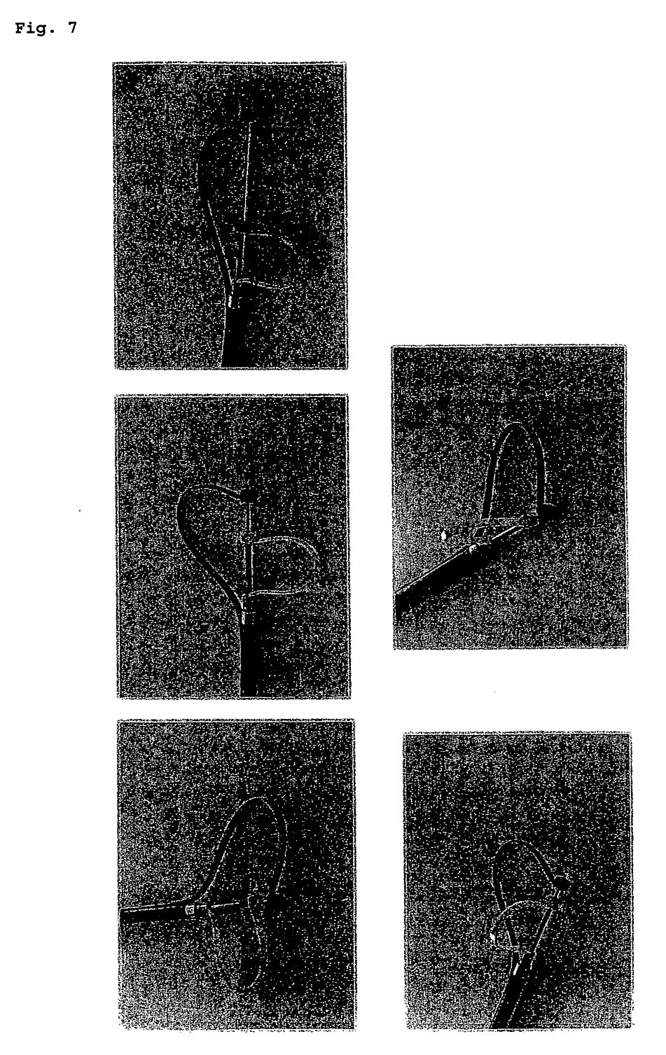

As shown in FIG. 6, according to another example of the present invention, the retractor may also be constituted such that the rod-shaped support member or the movable member is rotatable, or as shown in FIG. 7, such that the movable member operates under the system with two axes in order for the respective strip-shaped members to extend and retract independently. Meanwhile, the number of axes is not limited to two and the movable member may be operated under the system with three axes or more, depending on the number of strip-shaped members provided.

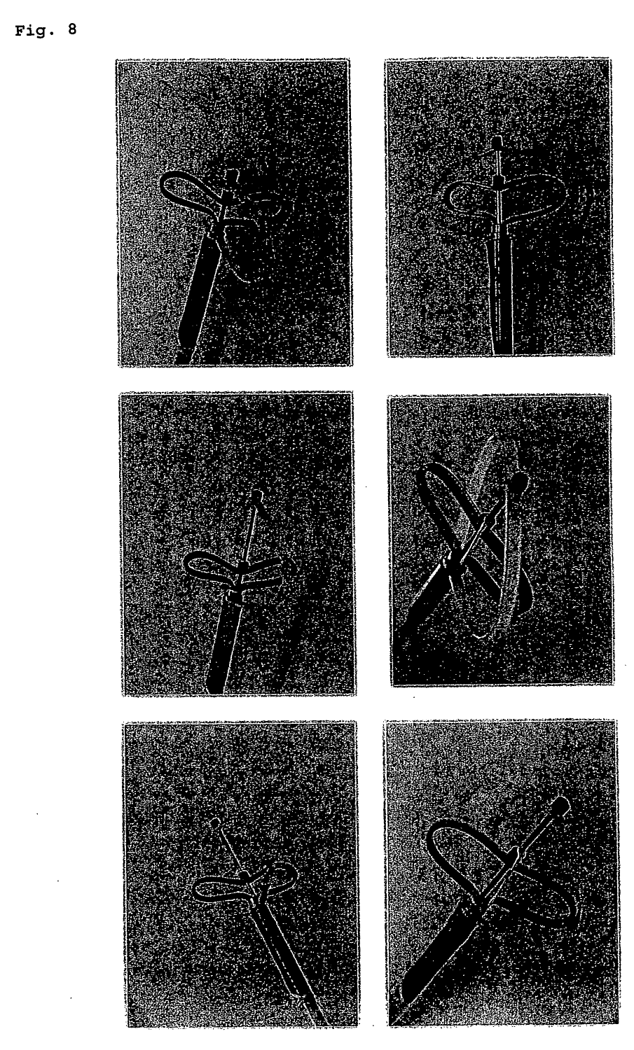

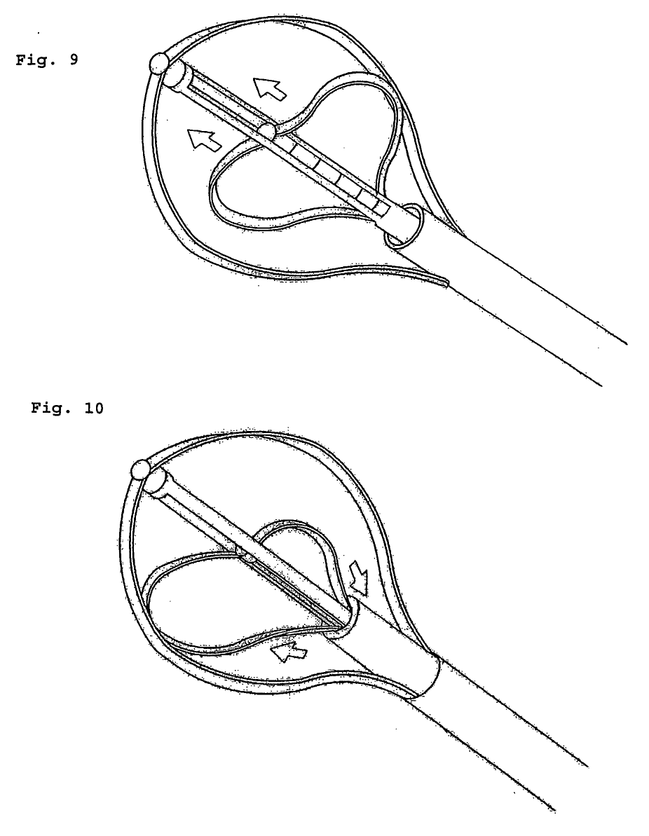

Further, the retractor according to an example of the present invention can be exemplified by the retractor illustrated in FIG. 8 and FIG. 9. Such retractors are provided with two pairs of strip-shaped members, wherein the strip-shaped members are respectively symmetrical relative to the movable member, and are constituted such that the two pairs of strip-shaped members are capable of extending, retracing, and rotating independently.

Still further, the retractor according to an example of the present invention can be exemplified by the retractor shown in FIG. 10 comprising a holding part formed by the strip-shaped members which can be independently altered in length respectively. In other words, it is a constitution wherein by fixing the other end of the strip-shaped member whose one end is fixed to the movable member, to a shifting member inserted in the interior of the rod-shaped support member, the length of the strip member forming the holding part can be altered, by causing the other end side of the strip-shaped member to go in and come out of the rod-shaped member according to the forward-and-backward movement of the shifting member.

### EXPLANATION OF LETTERS OR NUMERALS

10. retractor
12. holding part
14. rod-shaped support member
16. movable member
18. strip-shaped member (band-like member)
20. strip-shaped member (band-like member)
22. locking means
24. bulb-shaped member
26. gripping member

1. A retractor provided with a holding part for holding a living tissue, wherein the holding part comprises a movable member capable of a forward-and-backward movement protruding from the end part of a rod-shaped support member, and at least a pair of flexible strip-shaped members whose one end is fixed to the movable member; and wherein the strip-shaped members expand into an extended condition at the advancing-limit position of the movable member, and the pair of strip-shaped members form an asymmetric shape relative to the movable member at a given position of the movable member backward from the advancing-limit position.

2. The retractor according to claim 1, wherein the movable member is capable of moving in a full stroke.

3. A retractor provided with a holding part for holding a living tissue, wherein the holding part comprises a movable member capable of moving forward and backward in a full stroke, protruding from the end part of the rod-shaped support member, and a flexible strip-shaped members whose one end is fixed to the movable member; and wherein the strip-shaped member expands into an extended condition at the advancing-limit position of the movable member, and the strip-shaped member forms a curved shape relative to the movable member at a given position of the movable member backward from the advancing-limit position.

4. The retractor according to claim 3, wherein the strip-shaped members are at least one pair of strip-shaped members, and the pair of strip-shaped members form an asymmetric curved shape relative to the movable member at a given position of the movable member backward from the advancing-limit position.

5. The retractor according to any one of claims 1 to 4, wherein the rod-shaped support member and/or the movable member is rotatable.

6. The retractor according to any one of claims 1 to 5, comprising a locking means capable of locking the movable member at a given position.

7. The retractor according to any one of claims 1 to 6, wherein a bulb-shaped member is provided at the distal end of the holding part.

8. The retractor according to any one of claims 1 to 6, wherein an electrode is provided at the distal end of the holding part.

9. The retractor according to any one of claims 1 to 6, provided with a suction opening capable of suctioning body fluid.

10. A retractor provided with a holding part for holding a living tissue, wherein an electrode is provided at the distal end of the holding part.

11. A retractor provided with a holding part for holding a living tissue, comprising a suction opening capable of suctioning body fluid.
